(19) United States
(12) Patent Application Publication
(10) Pub. No.: US 2002/0026164 A1
(43) Pub. Date: Feb. 28, 2002

(54) Diaper with wetness detector

(76) Inventors: Alejandro Camarero Roy, Calatayud Zaragoza (ES); Fernando Camarero Roy, Calatayud Zaragoza (ES)

Correspondence Address: Evenson, McKeown, Edwards & Lenahan, P.L.L.C., 1200 G Street, N.W., Suite 700, Washington, DC, 20005, US

(21) Appl. No.: 09/813,972

(22) Filed: Mar. 22, 2001

(30) Foreign Application Priority Data
Aug. 30, 2000 (ES) 200002068

(51) Int. Cl. A61F 13/15

(52) U.S. Cl. 604/361

(57) ABSTRACT

A diaper has a flexible laminar body configured to a body of a baby and, in a central portion of a face thereof which is in contact with the baby's body, an area of wetness-absorbent material is provided. Attachments are arranged at ends of the laminar body for placing, attaching and removing of the diaper from the baby's body. In the central portion, humidity, thermal and electrochemical sensors are provided so that the thermal and electrochemical sensors sense the temperature and chemical analyses of the baby's urine. The sensors constitute an emitter that sends a signal to the receiver for emitting an acoustic and/or luminous signal.

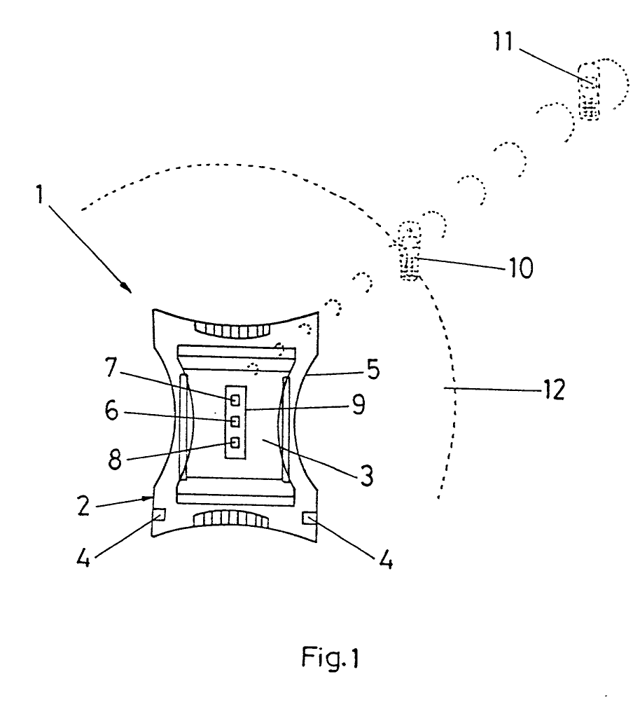

### BACKGROUND OF THE INVENTION

This application claims the priority of ES 200002068, filed Aug. 30, 2000, the disclosure of which is expressly incorporated by reference herein.

The present invention relates to a diaper having a wetness detector.

A large variety of diapers are known in the market which are differentiated only by their construction, since some have greater powers of liquid absorption than others. In other cases, the diapers are differentiated by the better or worse system of attaching them to the baby's body.

### SUMMARY OF THE INVENTION

An object of the present invention is to provide a diaper with an improved wetness detector.

The diaper of the present invention is personalized, and also has wetness and thermal sensors, and an electrochemical biosensor. These three sensors together constitute an emitter that sends a signal to a receiver-emitter than can start up a fixed or mobile telephone.

The analytical thermal detector determines the temperature and chemical analysis of the baby's urine.

During the manufacture of the diaper, a disposable thermal micro-electrode sensor and a disposable multiple electrochemical biosensor are included. The wetness sensor consists of a humidity detector which allows simple visualization to see if the baby is wet or not because the device changes color according to the wetness.

The diaper emitter group has a coverage radius defined by a radial distance between the diaper and a receiver and an emitter that at the same time start up a fixed or mobile telephone. The receiver emits a sound or syntony or a luminous signal that advises the person that the baby is wet.

The name of the baby can be included using an applicator with quick drying ink, in the diaper manufacturing line.

Up to now the sale of diapers is made in pharmacies, shops, supermarkets, etc. All the brands are in the same space and at the time for the potential customer, which gives the option of choice between one product and another. With the diaper of the invention, because it is personalized, sales will overtake the rest of the competition, including the competition that sells to governmental health organization which is can be the largest point of sale.

### BRIEF DESCRIPTION OF THE DRAWING

Other objects, advantages and novel features of the present invention will become apparent from the following detailed description of the invention when considered in conjunction with the accompanying drawings.

The sole FIGURE is a plan view of the diaper of the present invention in a laid-out condition.

### DETAILED DESCRIPTION OF THE DRAWING

The diaper of the invention, designated generally by numeral 1, is of the type formed by a piece 2 of a laminar nature that contains in the central area 3 a material that absorbs wetness. At the ends of the piece 2 are arranged attachment elements 4 to attach the diaper to the baby's body, as well as the adaptability itself to the body corresponding to the lateral rounded edges 5.

A humidity sensor 6, a temperature sensor 7 and an analytical chemical sensor 8 for the urine are arranged in the central part of the diaper 1. The three sensors 6, 7, 8 together constitute an emitter 9 that sends a signal to a receiver 10 (shown schematically) that can be the receiver-emitter of a mobile and/or fixed telephone 11. The emitter 10 establishes the coverage area 12 of the emitter itself.

The emitter which is placed in the diaper works as follows. When miction occurs in the baby, the circuit of the wetness detecting sensor is closed, the thermal micro-electrode circuit captures the temperature. At the same time, the electrochemical sensor 8 analyzes the urine and sends a signal with the data of temperature in degrees and the chemical analysis in percentages. That is, it will for example analyze glucose, lactose, urea, uric acid, cholesterol, etc.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. Diaper, comprising a flexible laminar body configured to a body of a baby and having in a central portion of a face thereof, in contact with the baby's body, an area of wetness-absorbent material, wherein means is operatively arranged at ends of the laminar body for placing, attaching and removing of the diaper from the baby's body; and in the central portion humidity, thermal, and electrochemical sensors are operatively arranged whereby thermal and electrochemical sensors sense temperature of the baby's urine and chemical analysis of the urine and the sensors comprise an emitter that sends a signal to the receiver that emits an acoustic and/or luminous signal.
